(19) United States
(12) Patent Application Publication
(10) Pub. No.: US 2023/0083060 A1
(43) Pub. Date: Mar. 16, 2023

(54) METHOD FOR PRODUCING HYDROGEN FROM PORK USING PHOTOSYNTHETIC ORGANISMS

(71) Applicant: Henan Agricultural University, Zhengzhou (CN)

(72) Inventors: Chaoyang LU, Zhengzhou (CN); Quanguo ZHANG, Zhengzhou (CN); Zhiping ZHANG, Zhengzhou (CN); Huan ZHANG, Zhengzhou (CN); Danping JIANG, Zhengzhou (CN); Yanyan JING, Zhengzhou (CN); Jianzhi YUE, Zhengzhou (CN); Yameng LI, Zhengzhou (CN); Yang ZHANG, Zhengzhou (CN)

(21) Appl. No.: 17/566,598

(22) Filed: Dec. 30, 2021

(30) Foreign Application Priority Data
Sep. 8, 2021 (CN) 202111048080.4

(51) Int. Cl. C12P 3/00; C12N 1/20

(52) U.S. Cl. CPC C12P 3/00; C12N 1/20

(57) ABSTRACT

A method for producing hydrogen from pork by using photosynthetic organisms includes: 1) mixing pork and trypsin, and adding a citric acid-sodium citrate buffer solution to a mixture of the pork and the trypsin; adjusting the pH of the citric acid-sodium citrate buffer mixed with the pork and the trypsin to neutral, to yield a neutral solution; adding a hydrogen-production medium and photosynthetic bacteria HAU-M1 in the late logarithmic phase to the neutral solution; and 2) placing a mixture of the neutral solution, the hydrogen-production medium, and the photosynthetic bacteria HAU-Ml in an incubator at 28-32° C. and a light intensity of 2800-3200 lux in the nitrogen atmosphere for hydrogen production.

### CROSS-REFERENCE TO RELATED APPLICATIONS

Pursuant to 35 U.S.C. § 119 and the Paris Convention Treaty, this application claims foreign priority to Chinese Patent Application No. 202111048080.4 filed Sep. 8, 2021, the contents of which, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND

The disclosure relates to a method for producing hydrogen from pork using photosynthetic organisms.

Hydrogen production by photosynthetic organisms is a method for converting organic matter into hydrogen. Pork is enzymatically hydrolyzed to produce amino acids and converted into hydrogen by photosynthetic bacteria, thus providing a guide for bio-safety disposal of carcasses from diseased pigs. Substrate concentration, cellulase loading, and degradation ability of photosynthetic bacteria are important factors that affect the substrate degradation rate and hydrogen production efficiency. Substrate concentration causes changes in concentrations of biomass and reducing sugar so as to affect the hydrogen production with photosynthetic organisms. Studies show increased substrate concentration increases the organic loading rate, thus enhancing hydrogen production efficiency. Excessive substrate facilitates formation of excessive volatile fatty acids and lowers the pH of the reaction solution, thus leading to the death of hydrogen-production microorganisms, and reducing hydrogen production efficiency.

### SUMMARY

The disclosure provides a method for producing hydrogen from pork by using photosynthetic organisms, the method comprising:

1) mixing pork and trypsin, and adding a citric acid-sodium citrate buffer solution to a mixture of the pork and the trypsin; adjusting a pH of the citric acid-sodium citrate buffer mixed with the pork and the trypsin to neutral, to yield a neutral solution; adding a hydrogen-production medium and photosynthetic bacteria HAU-M1 in the late logarithmic phase to the neutral solution; and

2) placing a mixture of the neutral solution, the hydrogen-production medium and the photosynthetic bacteria HAU-M1 in an incubator at 28-32° C. and a light intensity of 2800-3200 lux in a nitrogen atmosphere for hydrogen production.

In a class of this embodiment, the hydrogen-production medium in 1) comprises: 0.4 g/L NH₄Cl, 0.5 g/L K₂HPO₄, 2 g/L NaCl, 0.1 g/L yeast extract, 0.2 g/L MgCl₂ and 3.56 g/L sodium glutamate.

In a class of this embodiment, in 1), the photosynthetic bacteria HAU-M1 in the late logarithmic phase is obtained by: inoculating photosynthetic bacteria HAU-M1 into in a growth medium; and placing the growth medium in an incubator at 28-32° C. and a light intensity of 2500-3500 lux for 48 h.

In a class of this embodiment, the growth medium comprises: 0.5 g/L NH₄Cl, 0.1 g/L K₂HPO₄, 1 g/L NaCl, 0.5 g/L yeast extract, 0.1 g/L MgSO4.7H₂O, 2 g/L CH₃COONa, and 1 g/L NaHCO₃.

In a class of this embodiment, in 1), the method comprises mixing 1-9 g of the pork and the trypsin; adding 90-110 mL of 0.1 mol/L citric acid-sodium citrate buffer solution to the mixture of the pork and the trypsin and adjusting the pH of the citric acid-sodium citrate buffer mixed with the pork and the trypsin to neutral; and adding 40-60 mL of the hydrogen-production medium and 40-60 mL of the photosynthetic bacteria HAU-M1 in the late logarithmic phase to the neutral solution.

In a class of this embodiment, in 1), every one gram of pork is mixed with 0.1-0.2 g of trypsin; before mixing the pork with the trypsin, the method further comprises heating the pork at 121° C. and 0.17 MPa for 20 min; cooling heated pork to room temperature; and mincing with a meat mincer.

The following advantages are associated with the method of the disclosure: pork is used as the substrate for producing hydrogen by photosynthetic bacteria HAU-M1. The hydrogen production performance is assessed by analyzing hydrogen production kinetics, as well as characteristics of liquid and gas produced by photosynthetic bacteria. The energy conversion rate is calculated and liquid phase is used to explain the causes of changes in hydrogen production. When the substrate concentration is 25 g/L, the maximum hydrogen yield reaches 93.55 mL/g volatile solid (VS), and the maximum energy conversion rate is 5.54%. The results of the experiment provide a new reference for the harmless and resourceful treatment of abnormal dead animals.

### BRIEF DESCRIPTION OF THE DRAWINGS

FIGS. 1A-1C depict the characteristics of hydrogen produced from pork according to one embodiment of the disclosure; where FIG. 1A is a plot of hydrogen concentration versus substrate concentration; FIG. 1B is a plot of hydrogen production rate versus substrate concentration; and FIG. 1C is a plot of volume of cumulative hydrogen yield and hydrogen yield versus substrate concentration;

FIG. 2A-2C depict the characteristics of liquid produced from pork according to one embodiment of the disclosure; where FIG. 2A is a plot of pH of reaction solution versus substrate concentration; FIG. 2B is a plot of redox potential versus substrate concentration; and FIG. 2C is a plot of concentration of soluble metabolites versus substrate concentration; and

FIG. 3 is a plot of an energy conversion rate of hydrogen production from pork according to one embodiment of the disclosure.

### DETAILED DESCRIPTION

To further illustrate, experiments detailing a method for producing hydrogen from pork using photosynthetic organisms are described below. It should be noted that the following examples are intended to describe and not to limit the description.

Materials and Methods.

1.1. Photosynthetic bacteria HAU-M1, available from Henan Agricultural University, comprised 27 wt. % Rhodospirillum rubrum, 25 wt. % Rhodopseudomonas capsulata, 28 wt. % Rhodopseudomonas palustris, 9 wt. % Rhodobacter sphaeroides, and 11 wt. % Rhodobacter capsulatus.

Photosynthetic bacteria HAU-M1 were inoculated into in a growth medium and grew in an incubator at 30° C. and a light intensity of 3000 lux for 48 h to reach late logarithmic phase.

Compositions of growth medium and hydrogen-production medium are shown in Table 1.

TABLE 1
Compositions of growth medium and hydrogen-production medium

| Growth medium (g/L) | | Hydrogen-production medium (g/L) | |
|---|---|---|---|
| NH₄Cl | 0.5 | NH₄Cl | 0.4 |
| K₂HPO₄ | 0.1 | K₂HPO₄ | 0.5 |
| NaCl | 1 | NaCl | 2 |
| Yeast extract | 0.5 | Yeast extract | 0.1 |
| MgSO₄•7H₂O | 0.1 | MgCl₂ | 0.2 |
| CH₃COONa | 2 | Sodium glutamate | 3.56 |
| NaHCO₃ | 1 | | |

1.2. Pork, cut from a pig of diseases or accidental causes, was available from a slaughter house. The pork was cooked in an autoclave (DSX-280KB30, Shanghai ShenAn Medical Instrument Factory) at 121° C. and 0.17 MPa for 20 min. The pork was cooled to room temperature, minced with a meat mincer, and saved for experimentation. The pork contained about 62.08 wt. % water, 35.67 wt. % volatile solid, and 2.24 wt. % ash.

1.3. Effect of substrate concentration on the production of hydrogen rate: 1 g, 3 g, 5 g, 7 g, and 9 g of pork were added to reactors, respectively, followed by addition of 0.1 g of trypsin (200,000 u/g, Xiasheng Enzyme Biotechnology Co., Ltd., Beijing) per gram of pork. After mixing, 100 mL of 0.1 mol/L (pH 4.8) citric acid-sodium citrate buffer solution was added and solution was neutralized with 2 mol/L sodium hydroxide. 50 mL of hydrogen-production medium and 50 mL of photosynthetic bacteria HAU-M1 in the late logarithmic phase were added, sealed by a rubber stopper, and blown with nitrogen for 5 min to ensure absence of oxygen in the reactor. The reactor was placed in the incubator at 30° C. and a light intensity of 3000 lux for hydrogen production. The characteristics of liquids and gases in the reactor were measured and recorded every 12 hours.

Oxygen bomb calorimetry: the pork was dried at 75° C. for 48 h, crushed, and placed on a crucible. The crucible was placed onto a crucible holder of an oxygen bomb and an ignition wire was connected to the crucible holder.

1.4. Detection method: the gas generated in the reactor was collected by a gas sampling bag. The volume of the gas from the gas sampling bag was measured using a gas syringe. The concentrations of the hydrogen and soluble substances were determined by two gas chromatographs, respectively (6820 GC-14B and 7890B, Agilent Technologies, USA). The pH value and redox potential of the reaction solution were measured using PHS-3S meter and SX712 model ORP metre, respectively. The concentration of reducing sugar was measured using 721 spectrophotometer.

1.5. Analysis method: based on the maximum cumulative hydrogen yield, the Gompertz equation was used to determine the maximum potential cumulative hydrogen yield P, the maximum hydrogen production rate Rₘₐₓ, and delay period λ:

$$H = P exp \{ - exp [ \frac{{R}_{m} e}{P} ( λ - t ) + 1 ] \} . \qquad ( 1 )$$

The overall rate of hydrogen production was obtained from:

$${R}_{overall} = \frac{P}{( P / {R}_{m} ) + λ} \cdot \frac{1}{V} . \qquad ( 2 )$$

The energy conversion rate was calculated from:

$$E = \frac{{V}_{{H}_{2}} \times {Q}_{{H}_{2}}}{{Q}_{DH} \times m} \times 100 \% . \qquad ( 3 )$$

Definition of abbreviation in the equations were shown in Table 2.

TABLE 2
Definition of abbreviation

| Abbreviation | Definition |
|---|---|
| H | Cumulative hydrogen yield (mL) |
| P | Maximum potential cumulative hydrogen yield (mL) |
| Rₘ | Maximum hydrogen production (mL/h) |
| λ | Delay period (h) |
| t | Time |
| e | 2.718 |
| V | Effective volume of a rector (mL) |
| E | Energy conversion rate (%) |
| V_{H}_{₂} | Volume of hydrogen (mL) |
| Q_{H}_{₂} | Calorific value of hydrogen (12.86 J/mL) |
| Q_{DH} | Calorific value of pork (20423 J/g) |
| m | Dry weight (g) |

2. Results and Discussion

2.1. Effect of Substrate Concentration on Hydrogen Production Rate.

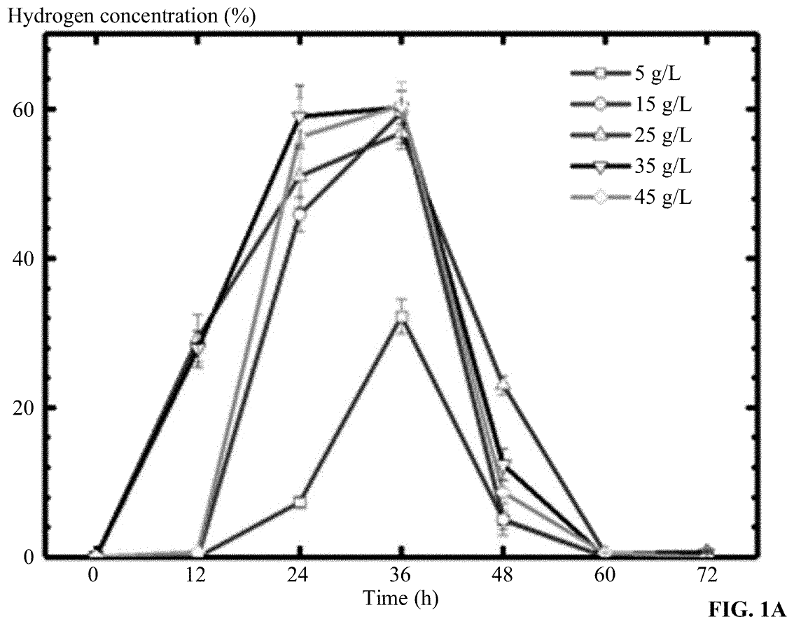

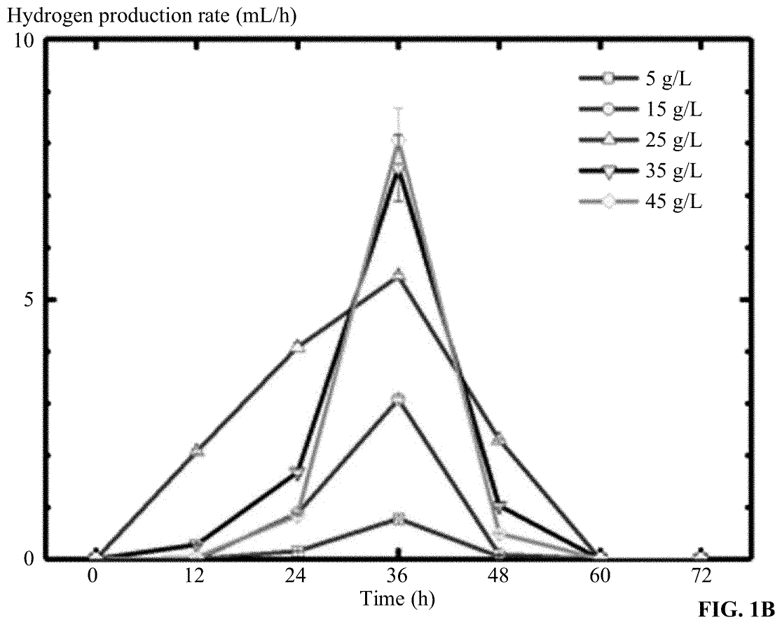

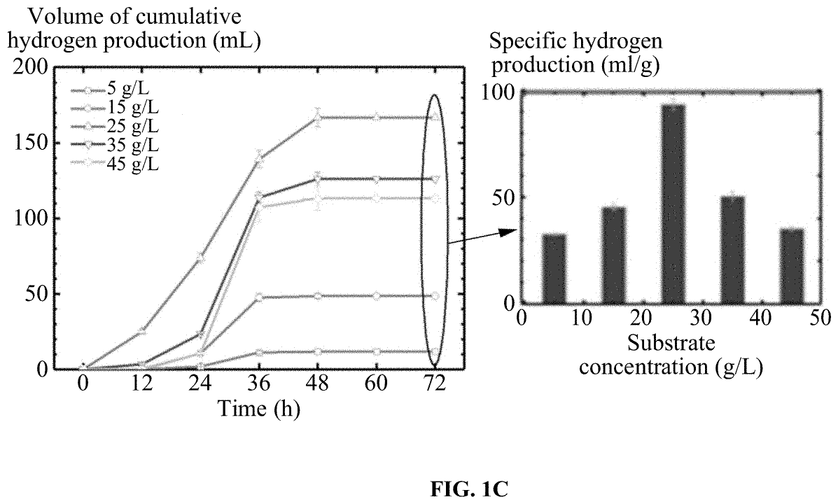

Results from these experiments are shown in FIG. 1A, the hydrogen concentration of the experimental group first increases and then decreases over time, which is consistent with main characteristics of the growth cycle of photosynthetic bacteria. 5 g/L of substrate results in low hydrogen production and the highest hydrogen concentration of 32.22% occurs at 36 h, which was 12 hours later than other experimental groups. The delay in the hydrogen production cycle is caused due to the lack of energy for photosynthetic bacteria with 5 g/L of substrate. With increase in substrate concentration, the photosynthetic bacteria quickly reach the peak of production of hydrogen. In the experimental group treated with 45 g/L substrate, the hydrogen production concentration reaches the peak of 60.48% at 36 h. The metabolism of photosynthetic bacteria is affected by the substrate concentration, resulting in a dose-dependent effects of hydrogen concentration on the substrate concentration. For example, the concentration of hydrogen produced by photosynthetic bacteria reaches the highest level when the substrate concentration is suitable.

The hydrogen production rate reflects the ability of photosynthetic bacteria to produce hydrogen in each time period. Referring to FIG. 1B, photosynthetic bacteria have a good ability to produce hydrogen within 24-36 h. In the experimental group treated with 45 g/L substrate, the highest hydrogen production rate was 8.06 mL/h at 36 h. Photosynthetic bacteria reached the late log growth phase at 36 h, exhibiting a higher growth rate and biological characteristics. The cumulative yield of hydrogen produced by photosynthetic bacteria is the area enclosed by the broken line of the hydrogen production rate and the X axis between 0-72 h. The peak hydrogen production rate reflects the increase in cumulative hydrogen production yield, as shown in FIG. 1C.

The term “cumulative hydrogen yield” used herein refers to the total amount of hydrogen gas produced in one reactor as of a particular time in the life of photosynthetic bacteria. Referring to FIG. 1C, the cumulative hydrogen yield increases with increasing substrate concentration in the range from 5-25 g/L. The substrate concentration continues to increase, leading to decreases in cumulative hydrogen yield. When the substrate concentration exceeds a threshold, photosynthetic bacteria obtain nutrients while producing more harmful substances, which inhibits the activity of photosynthetic bacteria. The results show that the decrease in cumulative hydrogen yield is caused by the substrate concentration that changes the characteristics of the reaction solution, such as pH value, redox potential, and volatile fatty acid concentration. The hydrogen production from the pork also first increased and then decreased, reaching the peak of 93.55 mL/g VS at 25 g/L.

As photosynthetic organisms break down pork to produce hydrogen, the pH value of the reaction solution first drops and then rises. The experimental group treated with a substrate concentration of 5 g/L exhibits the largest hydrogen production and the lowest pH 5.31. The results show that the substrate concentration is not positively related to the pH value of the reaction solution, and the change of the pH value is associated with the hydrogen production capacity of photosynthetic bacteria. As the pork is degraded into soluble fatty acids by photosynthetic bacteria, the pH of the reaction solution drops. As the soluble fatty acids are converted into hydrogen, the pH of the reaction solution rises.

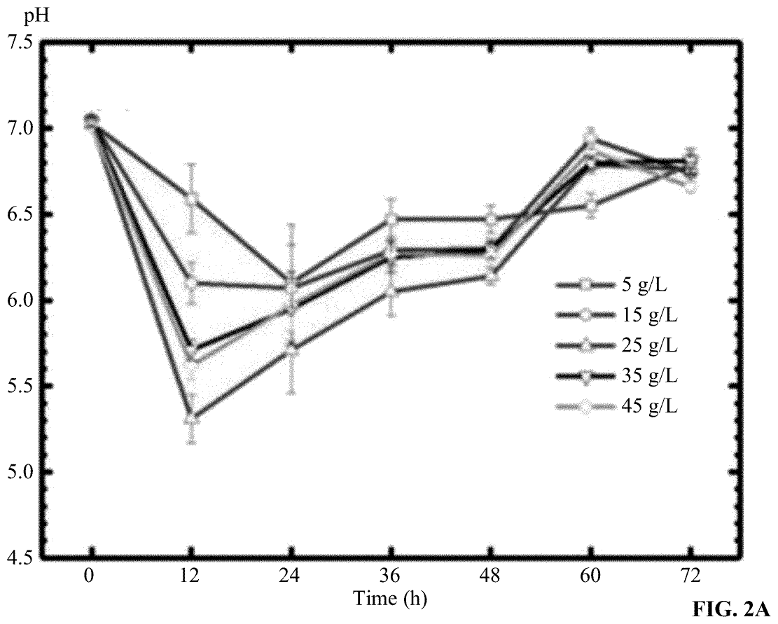

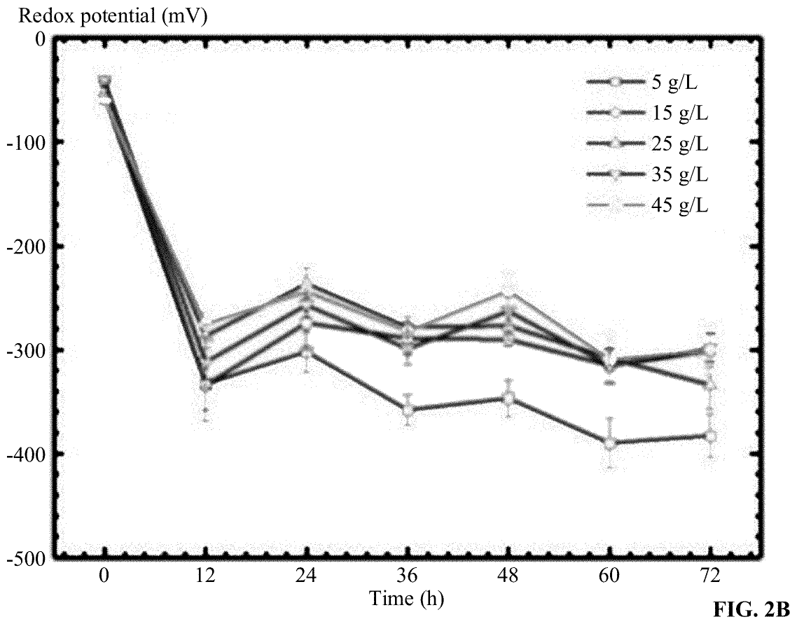

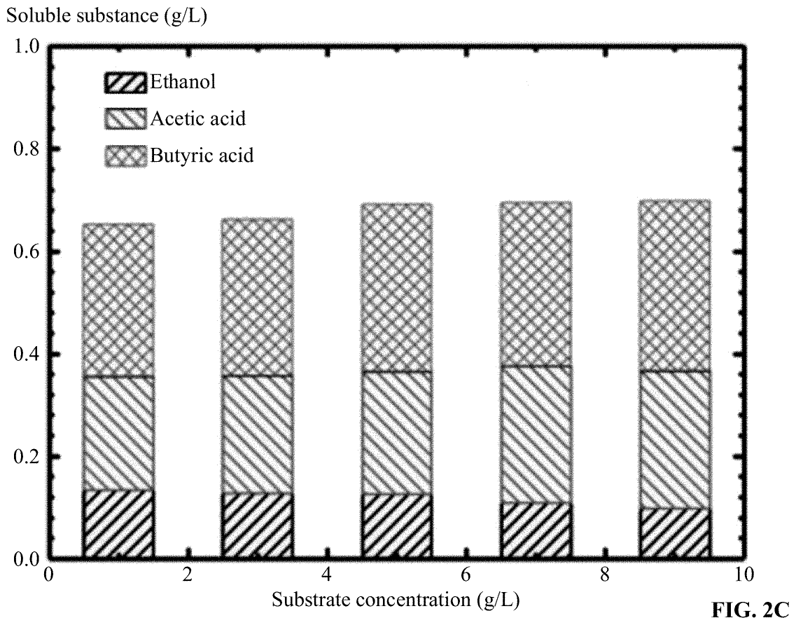

Results from these experiments are shown in FIG. 2B, as photosynthetic bacteria produce hydrogen from pork, the redox potential of the reaction solution declines to between −275 and −334 mV at 12 h and remains at low level. The reaction solution exhibits strong reducing properties, which facilitates the production of hydrogen by photosynthetic organisms. The hydrogen production in each experimental group tends to reach peak at a lower redox potential, so that the hydrogen production potential can be used to measure the ability of photosynthetic bacteria to produce hydrogen from pork.

Results from these experiments are shown in FIG. 2C, after the reaction is complete, the resulting liquid contains three soluble metabolites, including ethanol, acetic acid, and butyric acid. The resulting liquid is maximum in acetic acid and minimum in ethanol. The disparity in concentrations of the soluble metabolites among different experimental groups is little, which means the pH values of the final reaction solutions are basically the same. The research shows that pork is difficult to be degraded when exposed to excessive amount of soluble fatty acid. Excessive fatty acids will lead to the decrease of pH value, resulting in the decrease of bacterial activity.

TABLE 3
Hydrogen production kinetics

| Substrate | | | | | | Hydrogen |
|---|---|---|---|---|---|---|
| concentration | Pₘₐₓ | Rₘ | λ | | Rₒᵥₑᵣₐₗₗ | yield |
| (g/L) | (mL) | (mL/h) | (h) | R² | (mL/h) | (mL/g) |
| 5 | 11.72 | 1.32 | 22.75 | 0.9999 | 0.16 | 11.72 |
| 15 | 48.52 | 6.29 | 22.36 | 0.9999 | 0.67 | 16.17 |
| 25 | 173.42 | 5.84 | 9.95 | 0.9935 | 2.32 | 33.37 |
| 35 | 126.33 | 10.77 | 21.94 | 0.9997 | 1.75 | 18.00 |
| 45 | 113.29 | 13.10 | 23.58 | 0.9999 | 1.57 | 12.58 |

Kinetic analysis has important implications for simulating and predicting the kinetic characteristic of hydrogen produced from pork by photosynthetic bacteria. Results from these experiments are shown in Table 3, the kinetic prediction parameters are highly consistent with the actual experimental values according to the maximum potential cumulative hydrogen yield and the coefficient of determination R² greater than 0.99. When the substrate concentration is 45 g/L, the hydrogen production rate Rm reaches peak of 13.1 mL/h, indicating that the high substrate concentration facilitates the hydrogen production. When the substrate concentration is 25 g/L, photosynthetic bacteria have the shortest delay period, that is, the shortest delay in hydrogen production. When the substrate concentration is 25 g/L, the overall hydrogen production rate reaches peak of 2.32 mL/h. When the substrate concentration is 25 g/L, the hydrogen yield reaches peak of 33.37 mL/g, which is equal to 93.55 mL/g volatile solid (VS).

2.3 Energy Conversion Rate

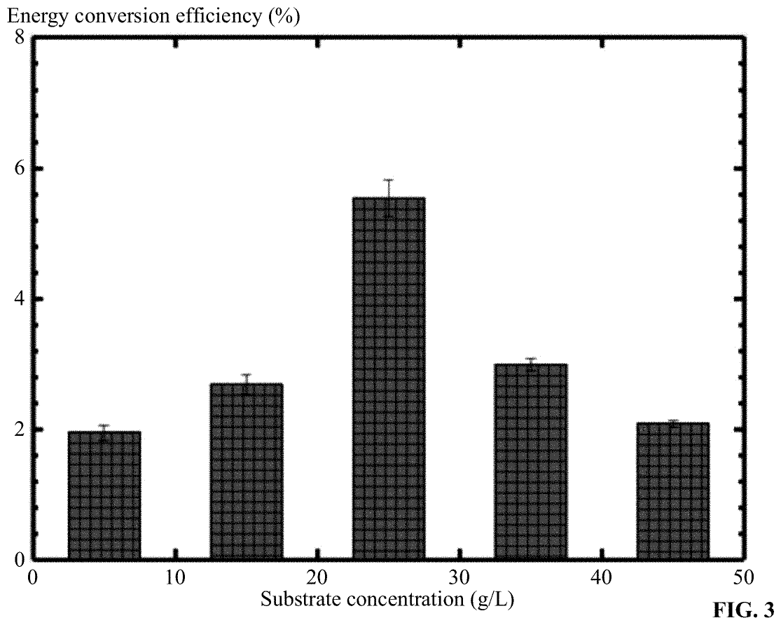

The term “energy conversion rate” used herein refers to an important indicator for evaluating the ability of pork to produce hydrogen gas. Referring to FIG. 3, the process of the energy conversion rate and the amount of pork meat trends consistently in all experimental groups, first increase and then decrease. When the substrate concentration is 25 g/L, the energy conversion efficiency reaches peak of 5.54%, which is lower than 9.84% of the hydrogen production from corn stover by photosynthetic biological (Lu C Y, Tahir N, Li W Z, Zhang Z P, Jiang D P, Guo S Y, et al. Enhanced buffer capacity of fermentation broth and biohydrogen production from corn stalk with Na₂HPO₄/NaH₂PO₄. Bioresource Technology. 2020; 313: 123783) and higher than the 4.14% reported by Liu et al (Liu H, Zhang Z, Zhang Q, Tahir N, Jing Y, Li Y, et al. Optimization of photo fermentation in corn stalk through phosphate additive. Bioresource Technology Reports. 2019; 7). The energy conversion efficiency various due to the different raw materials and methods.

Conclusion: The concentration of the substrate promotes the hydrogen production of photosynthetic organisms to show a similar normal distribution. When the substrate concentration is 25 g/L, the maximum hydrogen production reaches 93.55 mL/g VS, and the maximum energy conversion rate reaches 5.54%. The pH value drops first and then rises slowly, and the redox potential drops rapidly and then drops steadily. The kinetic parameters of hydrogen production are highly consistent with the actual parameters of the experiment. The results of the experiment provide a new reference for the harmless and resourceful treatment of abnormal dead poultry.

It will be obvious to those skilled in the art that changes and modifications may be made, and therefore, the aim in the appended claims is to cover all such changes and modifications.

What is claimed is:

1. A method, comprising:
1) mixing pork and trypsin, and adding a citric acid-sodium citrate buffer solution to a mixture of the pork and the trypsin; adjusting a pH of the citric acid-sodium citrate buffer mixed with the pork and the trypsin to neutral, to yield a neutral solution; adding a hydrogen-production medium and photosynthetic bacteria HAU-M1 in a late logarithmic phase to the neutral solution; and
2) placing a mixture of the neutral solution, the hydrogen-production medium, and the photosynthetic bacteria HAU-M1 in an incubator at 28-32° C. and a light intensity of 2800-3200 lux in a nitrogen atmosphere for hydrogen production.

2. The method of claim 1, wherein the hydrogen-production medium in 1) comprises: 0.4 g/L NH₄Cl, 0.5 g/L K₂HPO₄, 2 g/L NaCl, 0.1 g/L yeast extract, 0.2 g/L MgCl₂ and 3.56 g/L sodium glutamate.

3. The method of claim 1, wherein in 1), the photosynthetic bacteria HAU-M1 in the late logarithmic phase is obtained by: inoculating photosynthetic bacteria HAU-M1 into in a growth medium; and placing the growth medium in an incubator at 28-32° C. and a light intensity of 2500-3500 lux for 48 h.

4. The method of claim 3, wherein the growth medium comprises: 0.5 g/L NH₄Cl, 0.1 g/L K₂HPO₄, 1 g/L NaCl, 0.5 g/L yeast extract, 0.1 g/L MgSO4.7H₂O, 2 g/L CH₃COONa, and 1 g/L NaHCO₃.

5. The method of claim 1, wherein in 1), the method comprises mixing 1-9 g of the pork and the trypsin; adding 90-110 mL of 0.1 mol/L citric acid-sodium citrate buffer solution to the mixture of the pork and the trypsin and adjusting the pH of the citric acid-sodium citrate buffer mixed with the pork and the trypsin to neutral; and adding 40-60 mL of the hydrogen-production medium and 40-60 mL of the photosynthetic bacteria HAU-M1 in the late logarithmic phase to the neutral solution.

6. The method of claim 2, wherein in 1), the method comprises mixing 1-9 g of the pork and the trypsin; adding 90-110 mL of 0.1 mol/L citric acid-sodium citrate buffer solution to the mixture of the pork and the trypsin and adjusting the pH of the citric acid-sodium citrate buffer mixed with the pork and the trypsin to neutral; and adding 40-60 mL of the hydrogen-production medium and 40-60 mL of the photosynthetic bacteria HAU-M1 in the late logarithmic phase to the neutral solution.

7. The method of claim 3, wherein in 1), the method comprises mixing 1-9 g of the pork and the trypsin; adding 90-110 mL of 0.1 mol/L citric acid-sodium citrate buffer solution to the mixture of the pork and the trypsin and adjusting the pH of the citric acid-sodium citrate buffer mixed with the pork and the trypsin to neutral; and adding 40-60 mL of the hydrogen-production medium and 40-60 mL of the photosynthetic bacteria HAU-M1 in the late logarithmic phase to the neutral solution.

8. The method of claim 4, wherein in 1), the method comprises mixing 1-9 g of the pork and the trypsin; adding 90-110 mL of 0.1 mol/L citric acid-sodium citrate buffer solution to the mixture of the pork and the trypsin and adjusting the pH of the citric acid-sodium citrate buffer mixed with the pork and the trypsin to neutral; and adding 40-60 mL of the hydrogen-production medium and 40-60 mL of the photosynthetic bacteria HAU-M1 in the late logarithmic phase to the neutral solution.

9. The method of claim 5, wherein in 1), every one gram of pork is mixed with 0.1-0.2 g of trypsin.

10. The method of claim 6, wherein in 1), every one gram of pork is mixed with 0.1-0.2 g of trypsin.

11. The method of claim 7, wherein in 1), every one gram of pork is mixed with 0.1-0.2 g of trypsin.

12. The method of claim 8, wherein in 1), every one gram of pork is mixed with 0.1-0.2 g of trypsin.

13. The method of claim 9, wherein before mixing the pork with the trypsin, the method further comprises heating the pork at 121° C. and 0.17 MPa for 20 min; cooling heated pork to room temperature; and mincing the pork with a meat mincer.

14. The method of claim 10, wherein before mixing the pork with the trypsin, the method further comprises heating the pork at 121° C. and 0.17 MPa for 20 min; cooling heated pork to room temperature; and mincing the pork with a meat mincer.

15. The method of claim 11, wherein before mixing the pork with the trypsin, the method further comprises heating the pork at 121° C. and 0.17 MPa for 20 min; cooling heated pork to room temperature; and mincing the pork with a meat mincer.

16. The method of claim 12, wherein before mixing the pork with the trypsin, the method further comprises heating the pork at 121° C. and 0.17 MPa for 20 min; cooling heated pork to room temperature; and mincing the pork with a meat mincer.
